(19) United States
(12) Patent Application Publication
(10) Pub. No.: US 2025/0339634 A1
(43) Pub. Date: Nov. 6, 2025

(54) Device to Modulate Airway Clearance and Uses Thereof

(71) Applicants: Saurabh Biswas, College Station, TX (US); John C. Criscione, College Station, TX (US); Srujan Kancharla, Georgetown, TX (US)

(72) Inventors: Saurabh Biswas, College Station, TX (US); John C. Criscione, College Station, TX (US); Srujan Kancharla, Georgetown, TX (US)

(73) Assignee: The Texas A&M University System, College Station, TX (US)

(21) Appl. No.: 19/197,601

(22) Filed: May 2, 2025

Related U.S. Application Data
(60) Provisional application No. 63/641,469, filed on May 2, 2024.

(51) Int. Cl. A61M 16/00; A41D 1/04; A61B 5/00; A61B 5/0205; A61B 5/113; A61H 9/00; A61H 31/00; A61M 16/06

(52) U.S. Cl. CPC A61M 16/024; A41D 1/04; A61B 5/0205; A61B 5/4836; A61B 5/6803; A61H 9/0078; A61H 31/00; A61M 16/0006; A61M 16/0009; A61M 16/06; A41D 2400/00; A61B 5/1135; A61H 2201/107; A61H 2201/165; A61M 2205/70

(57) ABSTRACT

Provided herein are therapeutic devices effective to improve respiratory function and clear airways of a subject or patient. The therapeutic devices have at least one of a vest or wrap or an airway device, a power source, a driver operably connected to the power source configured to provide a driver action, a breathing event detector and a gating apparatus. The gating apparatus has a microcontroller and controls at least a relay, power to the driver or an output of the driver, a logical operation of the driver or a driveline component configured to operably interrupt or enable generation of and/or delivery of the driver action based on at least one detected breathing event. Also provided are methods for performing respiratory therapy on the subject via the therapeutic devices.

### CROSS-REFERENCE TO RELATED APPLICATIONS

This non-provisional patent application claims benefit of priority under 35 U.S.C. § 119(e) of provisional patent application U.S. Ser. No. 63/641,469, filed May 2, 2024, the entirety of which is hereby incorporated by reference.

### STATEMENT REGARDING FEDERALLY SPONSORED RESEARCH

This invention was made with government support under Grant Number 1644743 awarded by the National Science Foundation. The government has certain rights in the invention.

### BACKGROUND OF THE INVENTION

### Field of the Invention

The present invention relates generally to the fields of pneumatic devices and pulmonary medicine. More specifically, the present invention relates to airway clearance devices and uses thereof in respiratory diseases.

### Description of the Related Art

Presently, over 1,00,000 cystic fibrosis patients and 4 million bronchiectasis patients within 16 million COPD affected individuals have severe lung conditions where they need airway clearance to prevent serious lung inflammation and infections. Airway clearance therapies as high frequency chest wall oscillation is standard of care for treating cystic fibrosis and bronchiectasis.

To clear mucus from the airway of a patient, external oscillating pressure can be applied to the chest to enhance the airflow in the lungs so that mucus can be picked up and moved out of the system. Presently, FDA approved devices exist that automate this clearance process, making it more accessible and convenient and less expensive than the traditional manual chest physiotherapy performed by a trained clinician. The current approved device solutions, high-frequencey chest wall oscillation devices, primarily utilize pneumatic oscillation delivered through inflatable vests with portable or bedside pumps to assist in rapid compression and decompression. A second class of devices are the vibrational or percussion discs in vests that oscillate at a particular frequency with no pumps hence more portable. A third class of devices act on the airway rather than the chest wall with a mask covering the nose and/or mouth with suction applied intermittently by patient when coughing (e.g., cough assist device).

The chest-wall acting devices, however, apply pressure pulses for inflating and deflating as a constant waveform to the chest with no regard to the patient's natural breathing cycle. This results in poor clinical outcomes due to lack of patient compliance with regimen and decreased mobility. The continuous chest vibrations delivered by current devices can be a physical stressor to the patient. Providing oscillation during both inhale and exhale, dislodged mucus flows backwards into the lung during inhale and forward or out of the lungs during exhale. For the cough assist devices, the patient or therapist must activate the device at the appropriate time of the cough.

Thus, there remain unmet needs in the art for pneumatic devices configured for a specific subject or patient. More particularly, the art is deficient in airway clearance devices synchronized, automatically, with the respiration cycle of a patient during chest wall oscillation and/or cough assist therapy. The present invention fulfills this critical need and advancement in the art.

### SUMMARY OF THE INVENTION

The present invention is directed to a therapeutic device to improve respiratory function in a subject in need thereof. The device comprises a vest or wrap configured to apply oscillatory, mechanical stimulation to at least one of the subject's chest or abdomen or an airway device with a pneumatic connection to the subject's airway or a combination thereof and a power source. A driver is operably connected to the power source and is configured to provide a driver action comprising mechanical oscillation to the vest or wrap or suction to the airway device or a combination thereof. The device a breathing event detector. A gating apparatus is in electromechanical control of at least a relay, power to the driver or an output of the driver, a logical operation of the driver or a driveline component configured to operably interrupt or enable generation of and/or delivery of the driver action based on at least one detected breathing event. The present invention is directed to a related therapeutic device further comprising a manually operable push button in electronic communication with the breathing event detector and configured to enable calibration of the therapeutic device via the algorithm.

The present invention is also directed to a method for performing respiratory therapy on a subject in need thereof. In this method the vest or the wrap or the airway device or a combination thereof comprising the therapeutic device described herein is positioned on the subject and at least one breathing event is detected in the subject. The driver is activated to initiate the driver action comprising at least one of mechanical oscillation of the vest or the wrap or suction to the airway device and the driver is deactivated when the breathing event is no longer detected. Alternatively, a transmission of the driver action to the vest, the wrap or the airway device is gated when the driver action is continuous The present invention is further directed to a related method further comprising, prior to the positioning step, pushing a manually operable push button configured to calibrate the algorithm tangibly stored in the gating apparatus in the therapeutic device.

The present invention is directed further to an airway clearance device. The device comprises a vest with a plurality of inflatable air chambers therein and a power source. A driver comprising an air pulse generator is operably connected to the power source configured to inflate and deflate the plurality of inflatable air chambers in a mechanical oscillating driver action and an On/Off relay switch is in operable control of power to the driver. The airway clearance device has an anemometer in fluid connection with a mask placeable over the nose and mouth of the subject, said mask comprising at least one sensor therein and is configured to detect an exhalation breathing event. A gating apparatus comprises a microcontroller and is in electromechanical control of at least the On/Off relay switch, a logical operation of the driver power to the driver or an output of the driver, a logical operation of the driver or a driveline component configured to operably interrupt or enable generation of and/or delivery of the driver action based on at least one detected breathing event. The present invention is directed to a related airway clearance device further comprising a manually operable push button configured to enable calibration thereof.

The present invention is directed further still to a method for performing respiratory therapy on a subject in need thereof. In this method, the airway clearance device described herein is calibrated and the vest is positioned on the subject and pulses of air are delivered to at least one of the plurality of inflatable air chambers in the vest during voluntary breath exhalations, thereby mechanically oscillating the subject's chest to cause a therapeutic airway clearing effect thereto.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1B are flowcharts showing how the current state of the art (FIG. 1A) and the pneumatic device presented herein (FIG. 1B) are activated and deactivated.

FIGS. 2A-2B shows the air pressure detection device as placed on a subject wearing the therapeutic vest. FIG. 2A shows the air pressure detection device as a means to trigger the ON/OFF function of the pump. FIG. 2B is a simplified side view showing placement of the pressure detection device on the subject.

FIGS. 3A-3G illustrate additional means for detecting breathing events through airflow monitoring (FIG. 3A), acoustic monitoring of breath sounds (FIG. 3B), chest wall movement (FIG. 3C), imaging chest wall movement (FIG. 3D), cardiac activity via pulse wave velocity (FIGS. 3E-3G).

FIG. 4 is a schematic showing the electronic setup of the device.

FIG. 5A-5J illustrate the respiratory waveforms to activate and inactivate the airway clearance device.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

As used herein, the terms “subject” and “patient” are used interchangeably and refer to a human.

As used herein, the terms “airway clearance device” and “pneumatic device” are used interchangeably.

As used herein, the term “current device” refers to therapeutic devices currently known in the art.

As used herein, the terms “therapeutic vest”, “high-frequency chest wall oscillation vest or HFCWO vest” and “vest” are used interchangeably.

In one embodiment of the present invention, there is provided a therapeutic device to improve respiratory function in a subject in need thereof, comprising a vest or wrap configured to apply oscillatory, mechanical stimulation to at least one of the subject's chest or abdomen or an airway device with a pneumatic connection to the subject's airway or a combination thereof; a power source; a driver operably connected to the power source configured to provide a driver action comprising mechanical oscillation to the vest or wrap or suction to the airway device or a combination thereof; a relay in operable control of power to the driver; a breathing event detector; and a gating apparatus is in electromechanical control of at least a relay, power to the driver or an output of the driver, a logical operation of the driver or a driveline component configured to operably interrupt or enable generation of and/or delivery of the driver action based on at least one detected breathing event. Further to this embodiment the method comprises a manually operable push button in electronic communication with the breathing event detector and configured to enable calibration of the therapeutic device via the algorithm.

In both embodiments, the gating apparatus may comprise a microcontroller. Further to this embodiment the microcontroller may comprise a removable data storage card. The vest may be disposable or reusable. In addition, the vest may be portable. Also, the driver may be an air pulse generator or a cough assist device or a combination thereof.

In both embodiments, the breathing event detector may comprise at least one sensor that forms a closed feedback loop configured to sense a status of the respiratory function in the subject. In one aspect, the breathing event detector may comprise a mask containing the at least one sensor and placeable over nose and mouth of the subject and an anemometer in fluid connection with the mask. In another aspect, the breathing event detector may comprise a microphone placeable near the mouth of the subject in electronic communication with the air pulse generator. In yet another aspect, the breathing event detector may comprise a pulse wave velocity detector placeable on an arm of the subject and in electronic operable communication with the air pulse generator or the cough assist device or a combination thereof. In yet another aspect, the breathing event detector may comprise a chest band in electronic communication with the cough assist device. In yet another aspect, the breathing event detector may comprise an imaging scanner configured to scan the chest of the subject and in electronic communication with the cough assist device. In both embodiments and aspects thereof, the sensor may be configured to detect exhalation and inhalation, to detect breath sounds, to monitor air flow, to monitor chest expansion and contraction or to monitor cardiac activity and respiratory rate or a combination thereof.

In another embodiment of the present invention, there is provided a method for performing respiratory therapy on a subject in need thereof, comprising positioning on the subject the vest or the wrap or the airway device or a combination thereof comprising the therapeutic device described supra; detecting at least one breathing event in the subject; activating the driver to initiate the driver action comprising at least one of mechanical oscillation of the vest or the wrap or suction to the airway device; and deactivating the driver when the breathing event is no longer detected; or gating a transmission of the driver action to the vest, the wrap or the airway device when the driver action is continuous. Further to this embodiment, the method comprises, prior to the positioning step, pushing a manually operable push button configured to calibrate the therapeutic device.

In both embodiments, the breathing event for activation may be exhalation by the subject or chest and/or abdominal contraction upon the exhalation. Also, the breathing event for deactivation may be inhalation by the subject or chest and/or abdominal expansion upon the inhalation.

In one aspect of both embodiments, the activating step may comprise initiating the driver action when the breathing event is a start of an exhalation after a substantially deep inhalation. In another aspect, the deactivating step may comprise deactivating the driver when the breathing event is an inhalation below a lung volume threshold set for the subject. In yet another aspect, the activating step may comprise initiating the driver action when the breathing event is a substantially fast exhalation effort.

In yet another embodiment of the present invention, there is provided an airway clearance device, a vest with a plurality of inflatable air chambers therein; a power source; a driver comprising an air pulse generator operably connected to the power source configured to inflate and deflate the plurality of inflatable air chambers in a mechanical oscillating driver action; an On/Off relay switch in operable control of power to the driver; an anemometer in fluid connection with a mask placeable over the nose and mouth of the subject, said mask comprising at least one sensor therein configured to detect an exhalation breathing event; and a gating apparatus comprising a microcontroller, said gating apparatus in electromechanical control of at least the On/Off relay switch, power to the driver or an output of the driver, a logical operation of the driver or a driveline component configured to operably interrupt or enable generation of and/or delivery of the driver action based on at least one detected breathing event.

Further to this embodiment, the airway clearance device comprises a manually operable push button configured to enable calibration thereof. In another further embodiment, the microcontroller comprises a removable storage card. In all embodiments, the at least one sensor in the mask may be configured to enable a feedback loop comprising exhalation and inhalation.

In yet another embodiment of the present invention, there is provided a method for performing respiratory therapy on a subject in need thereof, comprising calibrating the airway clearance device described supra; positioning the vest on the subject; and delivering pulses of air to at least one of the plurality of inflatable air chambers in the vest during voluntary breath exhalations, thereby mechanically oscillating the subject's chest to cause a therapeutic airway clearing effect thereto. In this embodiment, the subject may have cystic fibrosis, bronchiectasis, chronic obstructive pulmonary disorder (COPD), or neuromuscular disorders.

In one aspect of this embodiment the calibrating step may comprise pressing continuously a push button electronically connected to an algorithm tangibly stored in the microcontroller and operably connected to the anemometer simultaneously with the subject's exhalation thereon; releasing the push button when the exhalation stops; transmitting readings from the anemometer acquired during the pressing and releasing of the push button to the algorithm; and calculating threshold values for the anemometer that govern toggling of the ON/OFF relay switch to operate the air pulse generator. In another aspect of this embodiment, the delivering step may comprise toggling the ON/OFF relay switch to ON when the subject's exhalations rotate the anemometer at a rate that exceeds the threshold value; and generating the pulses of air when the relay switch toggles to ON.

Provided herein are airway clearance devices or pneumatic devices, airway clearance systems and methods of use, for example, providing therapy personalized for the subject or patient in need of the device.

The device presented herein enables a personalized therapy where with the gated approach the pressure is delivered synchronized with a patient's inspirations, expirations or portion of the respiration cycles. This enables a therapy both effective in mucous clearance from the patient and in pressure management in the vest thereby providing a more comfortable therapy for the patient. The device delivers high-frequency chest wall oscillation to promote airway clearance and improve bronchial drainage in pediatric and adult patients who have acute and chronic respiratory diseases like cystic fibrosis, chronic obstructive pulmonary disease (COPD), and bronchiectasis. Typically high-frequency is in the range of about 10 Hz to about 50 Hz in current devices, thus high-frequency indicates herein higher than breathing frequency which is less than 1 Hz The device helps clear the lungs of excess secretions to help reduce respiratory infection and hospitalization risks for patients with a chronic lung conditions. The device utilizes the patient's physiological respiration states to gate the delivery of alternating air pulses into a vest garment which compresses and releases the chest wall, resulting in airflow oscillation in the airways. This movement acts to loosen, thin, and propel mucus toward major airways, where it can be expectorated.

Particularly, the airway clearance device or pneumatic device have the following components and features:
1. Gating: The driver/pump of the device is gated with the patient's own respiration cycle thus enabling the chest wall oscillation at a physiological pressure as compared to present devices that use a constant sinusoidal or triangular, or hybrid waveforms. This not only enhances the mucous clearance but also make the device much more comfortable for the patients to wear increasing the compliance of the therapeutic regimen. Considering pediatric patients with cystic fibrosis is the first indication pursued here these features make it adaptable to that patient population. A gating apparatus may comprise a microcontroller with a tangibly stored algorithm configured to receive input from a breathing event detector. The gating apparatus may have electromechanical control of 1) a relay, such as an ON/OFF switch, that controls power to the driver, 2) the logical operation of the driver, and/or 3) a driveline component of the driver, such as an automated value effective to either interrupt or to enable the generation of and/or delivery of the driver action to the therapeutic device or airway clearance device that are based on the detection of breathing events as described herein.
2. Integration with software and sensors: The designed vest has the ability to integrate with a digital management system for therapy compliance and monitoring. Moreover, the vest may be integrated with sensors, for example, but not limited to, acoustic sensors, to integrate closed feedback loop device that senses the mucous clearance status and activate the vest.
3. Algorithm: The algorithm calibrates itself via the use of a push button. In a non-limiting example, the user presses the push button when exhaling on the anemometer and stops pressing the button when not exhaling. The readings from anemometer and the values from the button when being pressed and not pressed are created into a .txt file that the microcontroller stores onto an SD card. This file is then analyzed by the microcontroller to determine the threshold values for when to toggle ON/OFF a relay switch, which turns ON/OFF the air pulse generator that simulates chest vibrations. After calibration, the patient continues to exhale onto the anemometer, which turns ON/OFF the medical device based on the determined threshold values. This continually runs till the device is manually turned off by the user.

The anemometer, relay switch, push button, and microcontroller with SD card are reusable. The vest worn by the patient may be reusable or disposable. The air pulse generator is commercially available. Improving portability of the airway clearance device may improve patient compliance.

Alternative features of the airway clearance device or pneumatic device are:
4. Non pneumatic ways of delivering chest wall oscillation: Vibrating discs in different configurations may be placed both front and back of the vest and are also actuated by the exhale of the patient to deliver the chest wall oscillation. That makes the device completely portable as it will negate the use of an external pump.
5. Detecting exhalation from the chest directly: The vest may be actuated via a direct mouth induced exhalation into a device and via an exhalation directly detected from the chest through sensor(s). In both cases, the exhalation triggers a signal to the pump leading to the actuation of the vest. Breathing events may be detected by using a tube (airway flow and/or airway pressure), using stretch sensors (chest-wall motion), using microphone (airway sounds), using ultrasound (lung volumes), and using bioimpedance (lung conductivity/density).

The airway clearance device provided herein is a high-frequency chest wall oscillation device (HFCWO) that enables delivery of an effective, personalized and home-based method for airway clearance therapy (ACT) for pediatric and adult patients with chronic respiratory conditions and neuromuscular disorders. During therapy, the device detects a patient's breathing pattern and selectively apply pressure pulses are applied selectively only during certain portions of the breathing cycle. This ensures that only forward airflow that moves mucus out of the system is enhanced, and not the backward” airflow that could potentially lodge the trapped mucus deeper in the lungs. This improves the safety and efficacy of treatment, making the experience much more comfortable for the patients as they are able to breathe normally throughout the process. Also, during a session of ACT, the vest design enables the application of pressure to the subject to be concentrated only in the most effective areas while avoiding the spine, breastbone, and other areas that could cause harm to the patient.

Embodiments of the present invention are better illustrated with reference to the Figure(s), however, such reference is not meant to limit the present invention in any fashion. The embodiments and variations described in detail herein are to be interpreted by the appended claims and equivalents thereof.

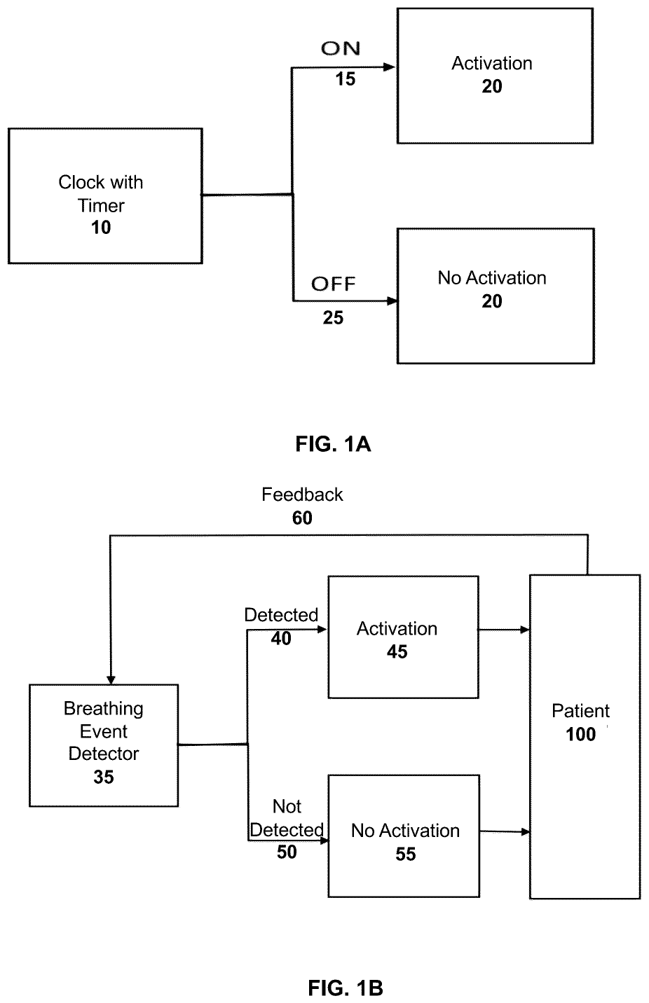

FIGS. 1A-1B are flowcharts illustrating generally the activation and deactivation of current devices and airway clearance devices, respectively. FIG. 1A shows that the current device, i.e., devices known in the art, utilizes a clock with a preset timer 10 which when ON at 15 results in activation 20 and when the timer turns OFF at 25 results in no activation 30. FIG. 1B shows the workflow of the airway clearance device. The breathing event detector 35 detects at 40 a breathing event in the patient 100 and activation 45 of the air clearance device occurs. If a breathing event that would activate the device is not detected at 50, no activation at 55 of the device occurs. Also, the breathing event detector via feedback 60 from the patient may inform the patient whether or not they are breathing correctly with activation or without activation.

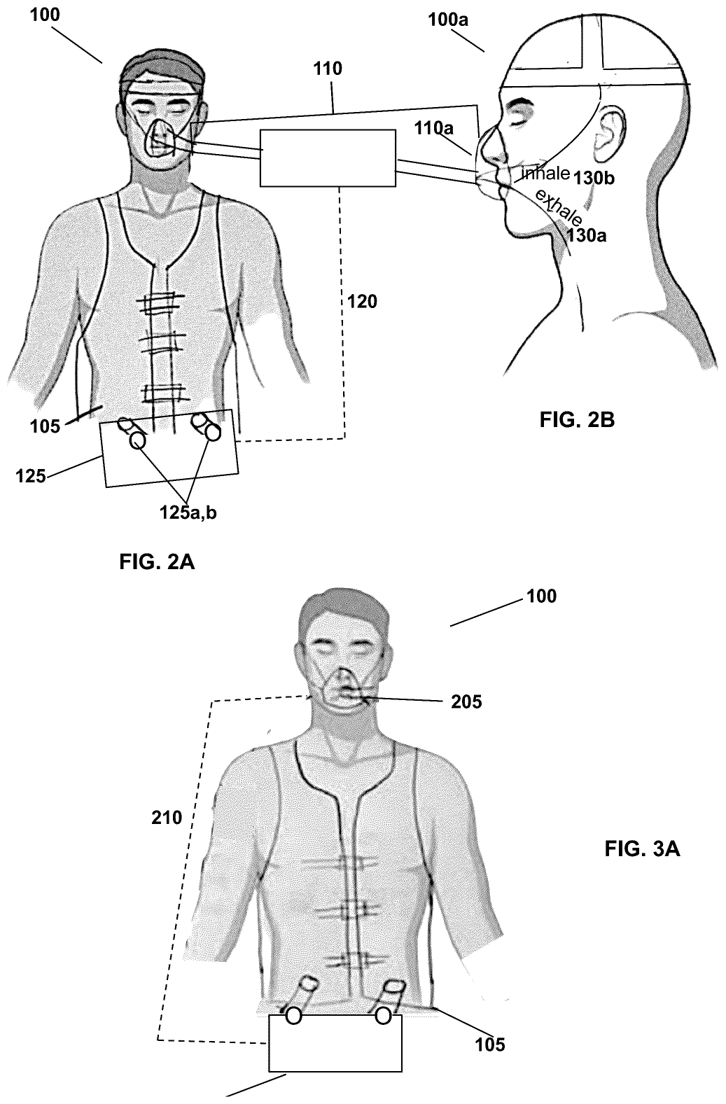

FIGS. 2A-2B illustrate the air pressure detector as placed on the subject and the electronic relationship with the airway clearance device. FIG. 2A shows the subject 100 wearing the therapeutic vest 105, for example, an HFCWO vest, and with the mask component 110a of the air pressure detector 110 or breathing event detector covering the nose and mouth of the subject. The mask has pressure sensors that sense pressure changes during exhalations and inhalations of the subject's respiratory cycle.

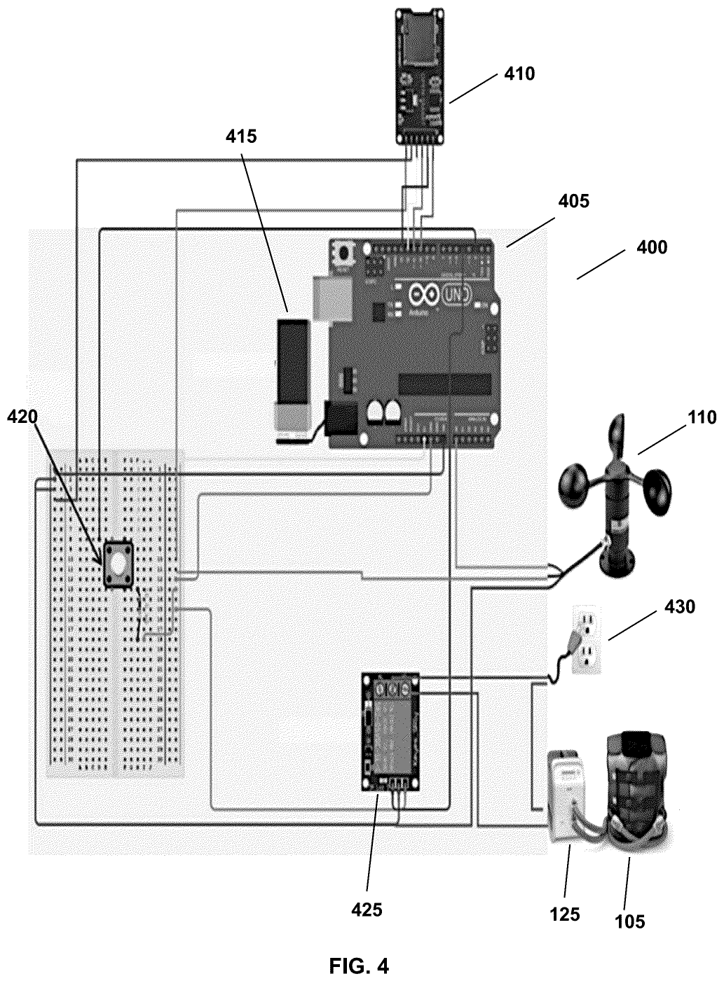

The air pressure detector may be an anemometer (see FIG. 4). The air pressure detector is in electronic communication at 120 with the pump 125 or air pulse generator (see FIG. 4). If the air pressure detector detects an exhalation, i.e., positive pressure, the pump is turned ON via a relay switch (see FIG. 4) to generate air pulses to inflate the vest at 125a,b thereby activating the airway clearance device. If the air pressure detector subsequently detects an inhalation, i.e., negative pressure, the relay switches to OFF (see FIG. 4), the pump ceases to generate air pulses, the vest is no longer inflated and the airway clearance device is deactivated.

With continued reference to FIG. 2A, FIG. 2B is a left side view of the subject's head 100a showing the mask 110a covering the mouth and nose. The passage of breath when the subject exhales 130a and inhales 130b is illustrated.

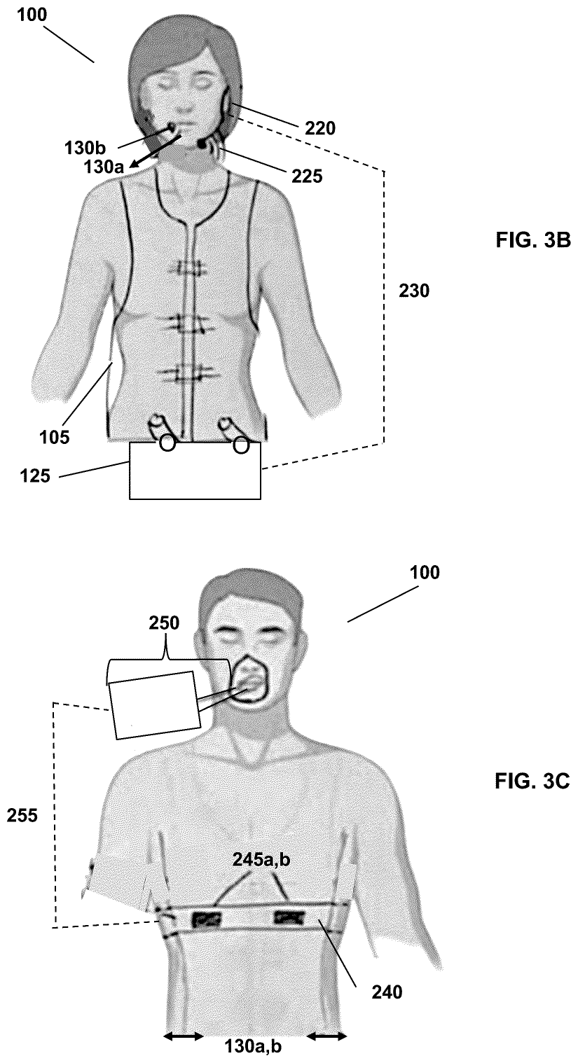

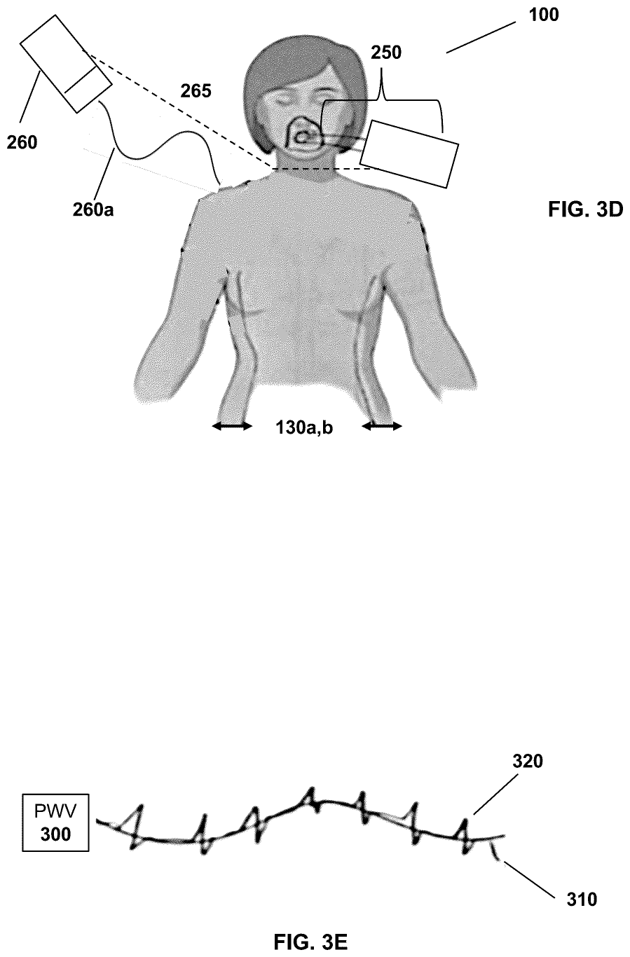

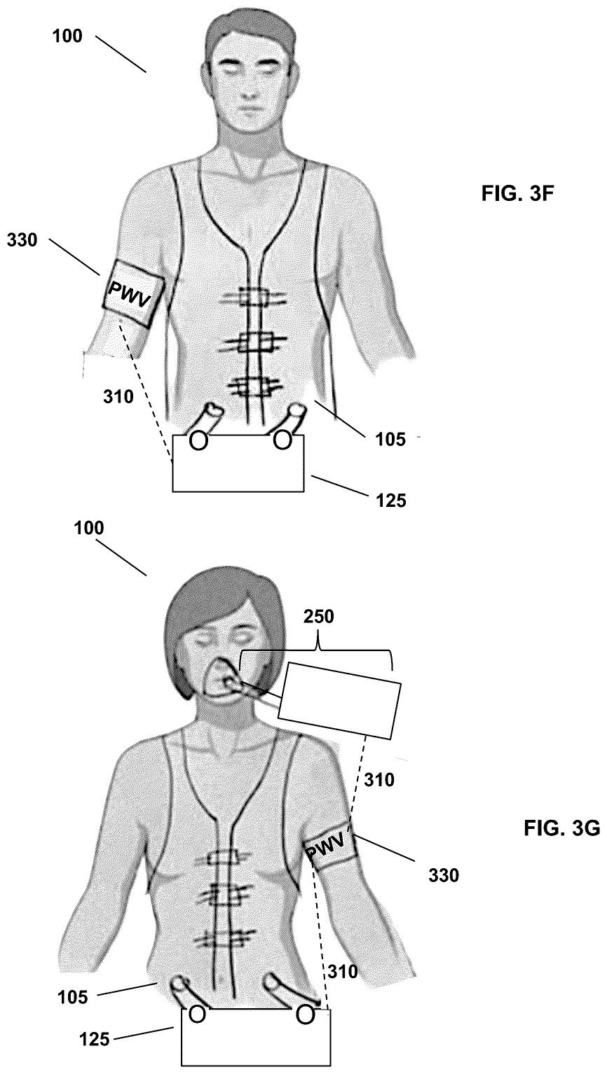

With continuous reference to FIG. 2A-2B, FIGS. 3A-3F illustrate alternative means for detecting breathing events. FIG. 3A illustrates detection of breathing events via airflow detection. The subject 100 is wearing an airflow detection mask 205 and the HFCWO vest 105. The airflow detection mask is in direct electronic communication at 210 with the pump 125 where the detection of positive airflow or exhalation turns on the pump via the relay switch to inflate the vest and during negative airflow or inhalation the relay switch flips to off and the pump is turned off.

With continued reference to FIG. 3A, FIG. 3B illustrates the detection of breathing events through acoustic monitoring of breath sounds which may be detected at various points along or adjacent to the respiratory track. The subject 100 is wearing the HFCWO vest 105 and a microphone 220, for example, hooked over the ear, which detects breath sounds 225 from the passage of breath when the subject exhales 130a and inhales 130b. The microphone is in direct electronic communication at 230 with the pump 125 which functions as described upon receipt of the acoustic signals from the microphone.

FIG. 3C illustrates the detection of breathing events by measuring chest wall movement. The subject 100 is wearing a chest band 240 that contains sensors 245a,b, such as, but not limited to, accelerometers. The sensors detect chest expansion and contraction associated with exhalation 130a and inhalation 130b, respectively. The chest band is in electronic communication with a cough assist device 250 which is activated upon detection of breathing events.

With continued reference to FIG. 3C, FIG. 3D illustrates the detection of breathing events by imaging chest wall movement. The subject 100 is wearing the cough assist device 250 while the chest wall is imaged at 255 via a LIDAR system 260 at 260a. The subject is imaged to detect chest expansion and contraction associated with exhalation 130a and inhalation 130b, respectively. The LiDAR system is in electronic communication at 265 with the cough assist device which is activated upon detection of breathing events.

FIG. 3E illustrates pulse wave velocity (PWV) 300. The respiratory rate waveform 310 is overlaid with the heart rate wave form 320.

With continued reference to FIG. 3E, FIG. 3F illustrates the detection of breathing events through cardiac activity. The subject 100 is wearing the HFCWO vest 105 and a pulse wave velocity (PWV) detector 330 on the arm. The PWV detector sends the respiratory rate waveform 310 to the pump 125 which is turned on and off via the relay to inflate the vest and activate and deactivate the airway clearance device as described.

With continued reference to FIG. 3F, FIG. 3G illustrates how multiple therapeutic activities may be triggered simultaneously. The subject 100 is wearing the HFCWO vest 105, the cough assist device 250 and the PWV detector 330. The PWV detector sends the respiratory rate waveform 310 simultaneously to the pump 125 to activate inflation of the vest and to the cough assist device.

FIG. 4 illustrates the electronic interconnections among the electromechanical gating apparatus and the airway clearance device components. The electromechanical gating apparatus 400 includes the microcontroller 405 with micro SD card 410 and battery power supply 415. The microcontroller electronically controls the push button 420 for calibration of the algorithm, the relay 425, for example, a switch and anemometer 110 or air pressure detector. The relay switch is in electrical connection with the driver, for example, an air pulse generator 125 at 430 which operates the therapeutic vest 105.

The following example is given for the purpose of illustrating various embodiments of the invention and are not meant to limit the present invention in any fashion.

### Example 1 Tracking Inhalation and Exhalation via Pressure Sensors: Operation Waveforms

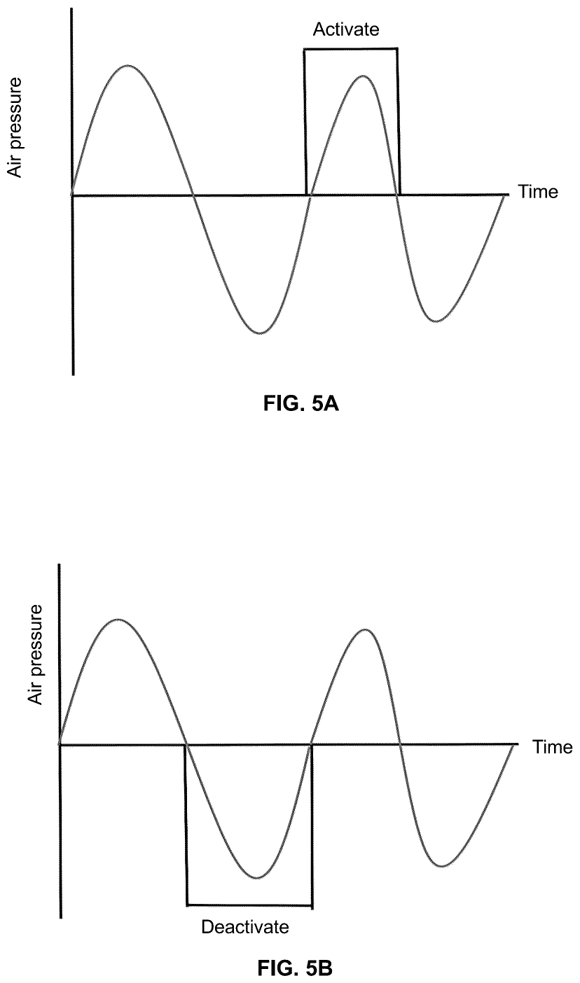

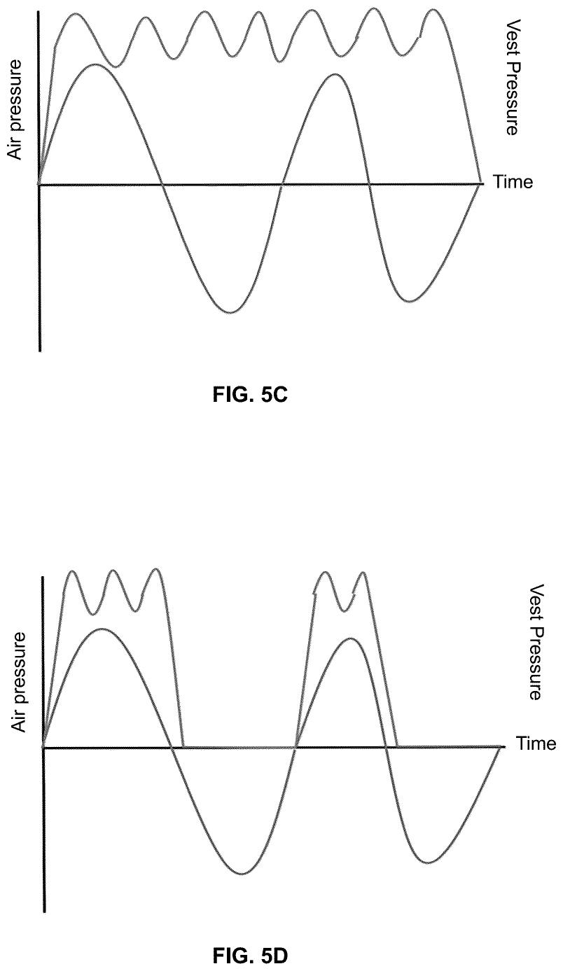

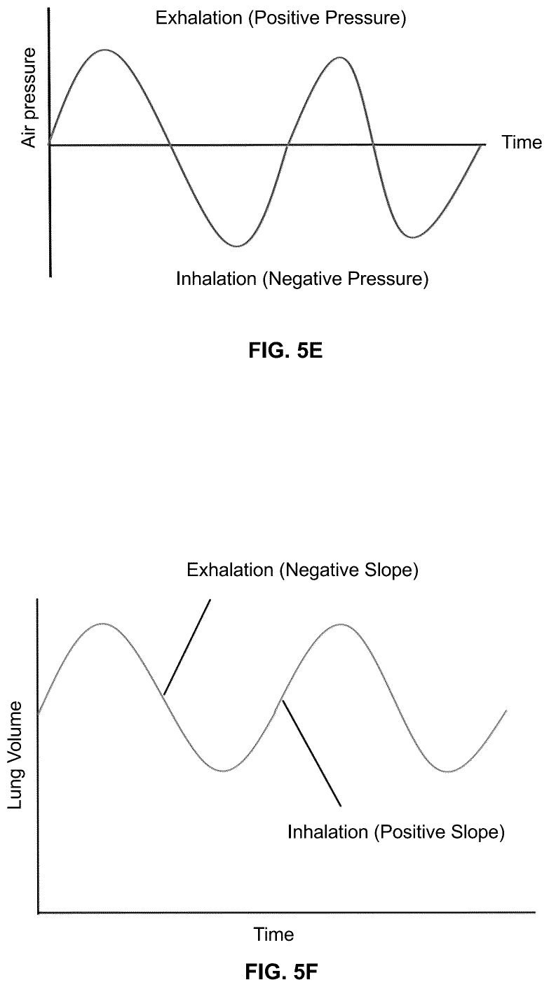

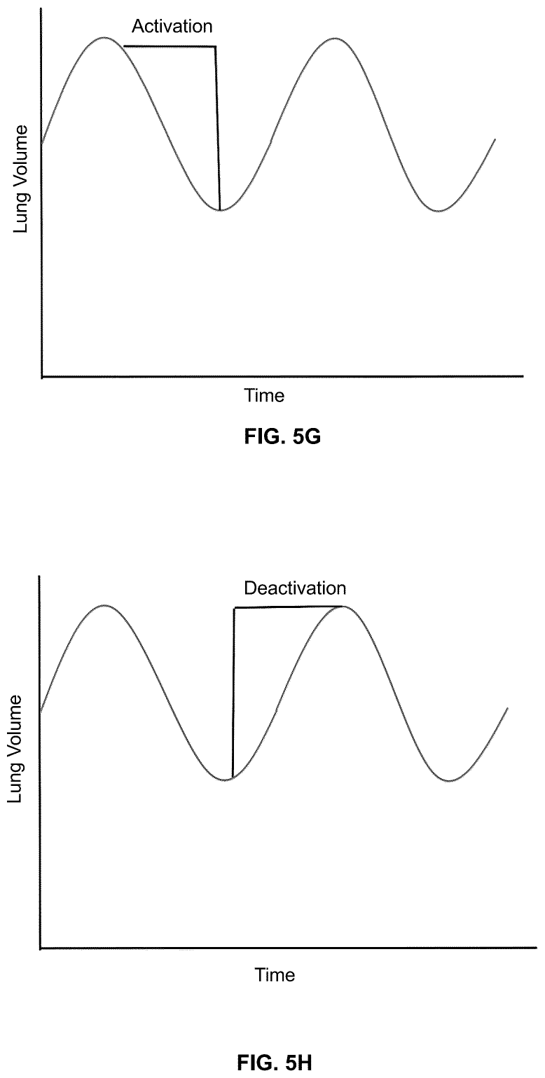

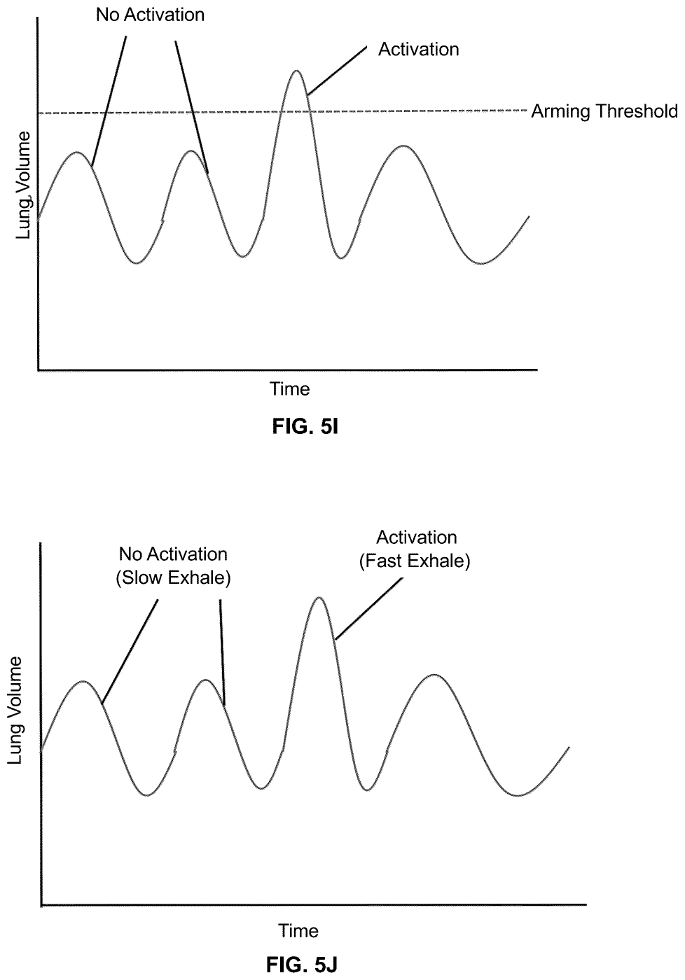

As shown in FIGS. 2A-2B, a breathing event detector enables detection of patient inhalations and exhalations. The breathing event detector is a pressure sensor placed inside a mask that a patient breathes through. Pressure sensors in masks can be used to track inhalation and exhalation of a respiratory cycle (U.S. Pat. No. 5,134,995A). Generally, assuming air pressure is measured at or near the patient's mouth or nose (FIGS. 2A-2B), the pressure is positive during exhalation and negative during inhalation (FIG. 5A). In another embodiment, where an air flow sensor replaces the pressure sensor, positive flow indicates exhalation, and negative flow indicates inhalation.

When a positive pressure or, alternatively, air flow rate, is detected by the pressure sensor, such as at the beginning of the patient's exhale cycle when air is exiting the lungs, an activation signal (FIGS. 5B-5C) is sent to the electromechanical gating apparatus, allowing pulsatile air flow from an air pulse generator to enter the vest. The pulsatile air flows into the vest, increasing pressure on the patient's thorax, and inducing a vibration to loosen mucus during the exhale phase. When the pressure sensors detect a negative pressure or air flow rate (FIG. 5D), the activation signal is terminated and the electromechanical gate blocks the flow of pulsatile air from the pulse air flow generator.

Monitoring lung volume or chest volume vs time in a subject also is indicative of exhalation and inhalation (FIG. 5E). A negative slope of the curve occurs when the volume is decreasing, and air is leaving the lung during exhalation corresponding to a period of activation of the airway clearance device (FIG. 5F). A positive slope occurs when air enters the lungs during inhalation corresponding to deactivation of the device (FIG. 5G).

During lung volume or chest volume monitoring, an arming threshold or trigger threshold may be set to activate the airway clearance device during exhalation after a deep enough inhalation has occurred (FIG. 5H). If the inhalation is below the lung volume threshold, the device does not activate during exhalation or will deactivate if already activated (FIG. 5I). A representative threshold may be set at the upper 50% of an exhalation event when the patient is instructed to breathe deeply.

The device may be activated during lung volume or chest volume monitoring during a fast exhale (FIG. 5J). A fast exhale is determined by how steep the negative slope of the lung volume vs time curve is. If the exhalation slope is not steep enough activation of the airway clearance device is blocked.

What is claimed is:

1. A therapeutic device to improve respiratory function in a subject in need thereof, comprising:
a vest or wrap configured to apply oscillatory, mechanical stimulation to at least one of the subject's chest or abdomen or an airway device with a pneumatic connection to the subject's airway or a combination thereof;
a power source;
a driver operably connected to the power source configured to provide a driver action comprising mechanical oscillation to the vest or wrap or suction to the airway device or a combination thereof;
a breathing event detector; and
a gating apparatus and in electromechanical control of at least a relay, power to the driver or an output of the driver, a logical operation of the driver or a driveline component configured to operably interrupt or enable generation of and/or delivery of the driver action based on at least one detected breathing event.

2. The therapeutic device of claim 1, further comprising a manually operable push button in electronic communication with the breathing event detector and configured to enable calibration of the therapeutic device.

3. The therapeutic device of claim 1, wherein the gating apparatus comprises:
a microcontroller.

4. The therapeutic device of claim 3, wherein the microcontroller further comprises a removable data storage card.

5. The therapeutic device of claim 1, wherein the driver is an air pulse generator or a cough assist device or a combination thereof.

6. The therapeutic device of claim 1, wherein the breathing event detector comprises at least one sensor that forms a closed feedback loop configured to sense a status of the respiratory function in the subject.

7. The therapeutic device of claim 6, wherein the breathing event detector comprises a mask containing the at least one sensor and placeable over nose and mouth of the subject and an anemometer in fluid connection with the mask.

8. The therapeutic device of claim 6, wherein the breathing event detector comprises a microphone placeable near the mouth of the subject.

9. The therapeutic device of claim 6, wherein the breathing event detector comprises a pulse wave velocity detector placeable on an arm of the subject.

10. The therapeutic device of claim 6, wherein the breathing event detector comprises a chest band.

11. The therapeutic device of claim 6, wherein the breathing event detector comprises an imaging scanner configured to scan the chest of the subject.

12. The therapeutic device of claim 6, wherein the sensor is configured to detect exhalation and inhalation, to detect breath sounds, to monitor air flow, to monitor chest expansion and contraction or to monitor cardiac activity and respiratory rate or a combination thereof.

13. The therapeutic device of claim 1, wherein the vest is disposable or reusable.

14. The pneumatic device of claim 1, wherein the vest is portable.

15. A method for performing respiratory therapy on a subject in need thereof, comprising:
positioning on the subject the vest or the wrap or the airway device or a combination thereof comprising the therapeutic device of claim 1;
detecting at least one breathing event in the subject;
activating the driver to initiate the driver action comprising at least one of mechanical oscillation of the vest or the wrap or suction to the airway device; and
deactivating the driver when the breathing event is no longer detected; or
gating a transmission of the driver action to the vest, the wrap or the airway device when the driver action is continuous.

16. The method of claim 15, further comprising, prior to the positioning step, pushing a manually operable push button configured to calibrate the therapeutic device.

17. The method of claim 15, wherein the activating step comprises initiating the driver action when the breathing event is a start of an exhalation after a substantially deep inhalation.

18. The method of claim 15, wherein the deactivating step comprises deactivating the driver when the breathing event is an inhalation below a lung volume threshold set for the subject.

19. The method of claim 15, wherein the activating step comprises initiating the driver action when the breathing event is a substantially fast exhalation effort.

20. The method of claim 15, wherein the breathing event for activation is exhalation by the subject or chest and/or abdominal contraction upon the exhalation.

21. The method of claim 15, wherein the breathing event for deactivation is inhalation by the subject or chest and/or abdominal expansion upon the inhalation.

22. An airway clearance device, comprising:
a vest with a plurality of inflatable air chambers therein;
a power source;
a driver comprising an air pulse generator operably connected to the power source configured to inflate and deflate the plurality of inflatable air chambers in a mechanical oscillating driver action;
an On/Off relay switch in operable control of power to the driver;
an anemometer in fluid connection with a mask placeable over the nose and mouth of the subject, said mask comprising at least one sensor therein configured to detect an exhalation breathing event; and
a gating apparatus and in electromechanical control of at least the On/Off relay switch, power to the driver or an output of the driver, a logical operation of the driver or a driveline component configured to operably interrupt or enable generation of and/or delivery of the driver action based on at least one detected breathing event.

23. The airway clearance device of claim 22, further comprising a manually operable push button configured to enable calibration thereof.

24. The airway clearance device of claim 22, wherein the microcontroller further comprises a removable data storage card.

25. The airway clearance device of claim 22, wherein the at least one sensor in the mask are configured to enable a feedback loop comprising exhalation and inhalation.

26. A method for performing respiratory therapy on a subject in need thereof, comprising:
calibrating the airway clearance device of claim 19;
positioning the vest on the subject; and
delivering pulses of air to at least one of the plurality of inflatable air chambers in the vest during voluntary breath exhalations, thereby mechanically oscillating the subject's chest to cause a therapeutic airway clearing effect thereto.

27. The method of claim 26, wherein the calibrating step comprises:
pressing continuously a push button electronically connected to an algorithm tangibly stored in the microcontroller and operably connected to the anemometer simultaneously with the subject's exhalation thereon;
releasing the push button when the exhalation stops;
transmitting readings from the anemometer acquired during the pressing and releasing of the push button to the algorithm; and
calculating threshold values for the anemometer that govern toggling of the ON/OFF relay switch to operate the air pulse generator.

28. The method of claim 26, wherein the delivering step comprises:
toggling the ON/OFF relay switch to ON when the subject's exhalations rotate the anemometer at a rate that exceeds the threshold value; and
generating the pulses of air when the relay switch toggles to ON.

29. The method of claim 26, wherein the subject has cystic fibrosis, bronchiectasis, chronic obstructive pulmonary disorder (COPD), or neuromuscular disorders.
